(19) United States
(12) Patent Application Publication
(10) Pub. No.: US 2009/0221940 A1
(43) Pub. Date: Sep. 3, 2009

(54) APPARATUS FOR TREATING BIOLOGICAL BODY SUBSTANCES BY MECHANICAL SHOCKWAVES

(75) Inventors: Ernst H. Marlinghaus, Bottighofen/TG (CH); Pavel Novak, Stetten/SH (CH); Josef Katona, Radolfzell (DE)

Correspondence Address: WOOD, PHILLIPS, KATZ, CLARK & MORTIMER, 500 W. MADISON STREET, SUITE 3800, CHICAGO, IL, 60661, US

(73) Assignee: Storz Medical AG, Tagerwilen (CH)

(21) Appl. No.: 12/393,699

(22) Filed: Feb. 26, 2009

(30) Foreign Application Priority Data
Feb. 29, 2008 (EP) 08003840.9

(51) Int. Cl. A61B 17/225

(52) U.S. Cl. 601/4

(57) ABSTRACT

The present invention relates to an apparatus for the treatment of biological body substances, in particular of the human body, by accelerating a projectile (13) and colliding it onto an impact body (9) which couples a mechanical shockwave to biological body substance. Therein, the impact body (9) is made of sintered ceramics.

### FIELD OF THE INVENTION

This invention generally relates to an apparatus and to a method for treating biological body substances by mechanical shockwaves.

### BACKGROUND OF THE INVENTION

Similar apparatuses are known, in particular, in the field of lithotripsy. There, body concrements, in particular, stones in the body tissue, are disintegrated by focused mechanical shockwaves. Besides the production by electrical discharges in water, apparatuses have been developed producing the mechanical shockwaves by the collision of an accelerated projectile and an impact body and coupling said shockwaves to body tissue by means of said impact body. Such apparatuses have also been used in lithotripsy by a direct contact between the impact body or a probe connected to the impact body and the stone, and in other treatments of biological body substances. In particular, these apparatuses are used for the treatment of muscle diseases and of diseases in the transition region between muscles and bones.

An example of an apparatus of the latter type is shown in EP 0 991 447. There, unfocused shockwaves shall be coupled to the body tissue.

### BRIEF SUMMARY OF THE INVENTION

An object of the invention is to provide an improved apparatus for treatment of biological body substances having a movable projectile and an impact body for producing a mechanical shockwave.

The invention provides an apparatus for treatment of biological body substances by being positioned onto a patient, said apparatus comprising a moveable projectile and an impact body, and being adapted to couple a mechanical shockwave to said biological body substance by accelerating said projectile and colliding said projectile onto said impact body, wherein said impact body is made of sintered ceramics, and a corresponding production method as well as a method of using such an apparatus.

Preferred embodiments are defined in the dependent claims.

A basic idea of the invention is to substitute metal collision bodies already known in the prior art, wherein in particular stainless steel has been used, by an advantageous material, namely sintered ceramics. Ceramics do not only have a very good biocompatibility and particularly low risks of allergies. Surprisingly, it has also been found that sintered ceramics can have a sufficient impact resistance and hardness for the application as an impact body, and do not crack or disintegrate, as originally assumed, after repeated collisions.

This leads to the opportunity to take advantage of different positive properties of certain ceramics depending on the individual application. Beside the already mentioned bio-compatibility this applies to the usually substantially lower heat conductivity compared to metals, as well. The patient subjectively feels a warmer and thus more comfortable and less unfamiliar appearing object, at least in case of a direct contact to the skin.

Further, many ceramics are much lighter than usual metals, in particular stainless steel. This has the advantage that the projectile, which is usually smaller due to reasons of construction, has a lower mass difference to the impact body in case of a common metallic implementation of the projectile so that the collision conditions can be improved in the sense of an increased transfer of impulse and energy. Further, also somewhat larger strokes of the impact body can be achieved that can be of interest.

Finally, many ceramics have advantageous acoustical impedances with less differences to typical acoustical impedances of body tissue compared to metals, in particular stainless steel. Substantially, this is the result of the lower mass density but also depends on the sound velocity.

In individual applications of the invention, the above-mentioned advantages need not be realized completely. Instead, the invention provides the opportunity to choose the one or the other positive and advantageous property of ceramics.

The term “ceramics” shall mean a material made of inorganic fine-particular raw materials being sintered i. e. having undergone a tempering step. Particularly preferred are oxides, especially metal oxides, carbides, in particular metal carbides and nitrides as well as mixtures thereof. Considered are for example zirconium oxide or silicon nitride. Preferred is a relative ratio of these materials of at least 80 weight-%, even better of 85 weight-%, 90 weight-% or even 95 weight-%.

Other constituents can also be included. In particular, in a certain ratio of at most 20 weight-%, preferably at most 15 weight-% or 10 weight-% or 5 weight-%, metallic constituents can be present, i. e. metallic particles or powders. In the area of powder metallurgy, similar working technologies as ceramic sintering are known for metals. By observation of the mentioned values, the positive properties of the ceramics will not be substantially decreased by these metallic additions. In the most preferred case, however, no metallic constituents are present at all.

An advantageous and already proven impact body geometry is substantially cylindrical, that is at least dominantly rotationally symmetrical, and has an entering surface to the projectile side and an exiting surface to the body side respectively at least intersecting the cylinder axis perpendicularly, preferably being generally perpendicular to the cylinder axis. They can also be slightly curved, in particular convex. The circumferential surface can have steps or a non-constant radius for various reasons. In particular, it can have structures e.g. a flange for bearing at least one elastomer ring.

Besides elastomer rings, other types of springs, such as helical springs, can also serve for decoupling the impact body from the remaining apparatus casing. Considered are also corrugated hoses or similar constructions having elastical properties.

A further preferred geometry of the impact body is based on a symmetrical ellipsoid of revolution symmetrical with regard to a longitudinal axis parallel to the movement direction of the projectile, which ellipsoid is shortened at the body substance side at least up to the focus of the ellipsoid of revolution to the body substance side, and on the projectile side at least up to the focus to the projectile side. In this embodiment, focused shockwaves shall be produced in a comparatively simple manner by a collision of the projectile onto the impact body and be coupled to the body. Hereto, the impact body shall serve as focusing element for converging the shockwaves produced therein by the collision of the projectile. This can be achieved by a ellipsoid of revolution as an impact body, the longitudinal axis of which shall be parallel to the movement direction of the projectile or correspond thereto.

For producing and coupling-in the shockwave, the ellipsoid of revolution is shortened on the projectile side such that the projectile impinges thereon in the respective focus. Consequently, the circumferential surface portions corresponding to the ellipsoid of revolution focus the shockwave onto the second focus. The impact body is shortened at this body substance side such that this focus is either directly on the body surface in contact to the impact body or is spaced therefrom in the body. Thus, by the ellipsoid of revolution shape, a focused shockwave can be coupled to the body. By means of the amount of shortening, an adjustment can be made whether the focus is positioned on the skin surface, in the skin region, or deeper in the tissue, depending on which body region shall be treated.

The ellipsoid of revolution allows a compact and, as regards its construction and handling, practical shape of the impact body being easily mounted, and offers besides that a focusing means without running time differences between the different focus portions. Namely, within an ellipse, the “rays” through a focus are reflected at the elliptical line such that they run through the other focus wherein various variants between both focuses have identical path length.

Preferably, the impact body is shortened at the projectile side and the side facing the body in a dull manner.

Pneumatic drives are preferred which can provide sufficient driving powers and accelerations for the projectile by a small technical effort. For example, advantageous speeds of the projectile in the range between 5 m/s to 50 m/s can be achieved thereby. A repetition operation is preferred, namely with preferred frequencies between 1 Hz and 50 Hz.

In contrast to embodiments of the prior art mentioned in the introduction, it can be preferred in this invention to work with relatively large strokes, that is macroscopic lateral displacements of the impact body compared to the overall apparatus. Herein, values of at least 0.5 mm, preferably at least 0.6 mm, can be chosen in particular. It has been found that the shockwave energy essential for many applications is actually coupled to the tissue to be treated by the macroscopic stroke movement of the impact body and that the acoustical shockwave produced and propagating in the impact body (which is thus not related to a macroscopic movement of the center of mass of the impact body) is in many cases not essential. In this sense, a sufficient stroke enables an effective coupling of the shockwave energy.

Finally, certain parameters of the ceramics used are preferred, in particular a relatively low density of preferably below 6 g/cm³, more preferably below 5 g/cm³ and even more preferably below 4 g/cm³. A low density reduces the mass of the impact body and thus the mass (preferably to be handled manually) of the mobile part of the apparatus. Also, it reduces the already mentioned acoustical impedance in an advantageous manner. Finally, it enables a certain size of the impact body without too big mass differences between the impact body and the projectile.

Further preferred is a so-called impact resistance of the ceramic material of at least 3,000 kJ/m², more preferred 4,000 kJ/m² and even more preferred 5,000 kJ/m² or more. This quantity determines the intensity with which the projectile may collide with the impact body without endangering the impact body itself.

Finally, relatively hard materials are preferred, in particular those having a larger pressure resistance of above 2,000 MPa.

Further, ceramics offer the opportunity to almost arbitrarily color the material without problems. Beside decorative aspects, this can be used in an advantageous manner for distinguishing different impact body types. In many applications, different collision bodies are provided to the user, which can be exchanged in the apparatus. By a color coding, errors are less probable than by using an alphanumerical characterization (which naturally can be provided additionally).

Further, the application of the apparatus for the treatment of soft body tissue, for example muscles or tendons, is particularly preferred. This includes the treatment of regions near to the bones and shockwave acupuncture. Typical indications are insertion tendonitis and other applications in orthopaedics and surgery as shoulder calcifications, heel pain, pseudarthroses, but also muscle pain. Further indications are in neurology such as the improvement of the mobility after strokes, the treatment of post-traumatic spasm and polyneuropathies. Within urology, for example chronic pelvic pain syndrome can be treated; in angiology/dermatology and surgery also scars or skin burns can be treated as well as improvements of wound healing can be reached.

As regards the production of the ceramic impact body, sinter methods are preferred in which the blank or the impact body in production is subjected to pressure. This can be done before and/or after tempering. In particular, an isostatical re-densification under heat can be performed.

### BRIEF DESCRIPTION OF THE DRAWINGS

FIG. 1 shows a schematic section along the longitudinal axis of a first embodiment of the invention.

FIG. 2 shows an analogous section through a second embodiment.

### DETAILED DESCRIPTION OF THE INVENTION

In the following, the invention will be explained making reference to exemplified embodiments, wherein the individual features can also be relevant for the invention in other embodiments, and which relate to the apparatus, the use, and the methods in general.

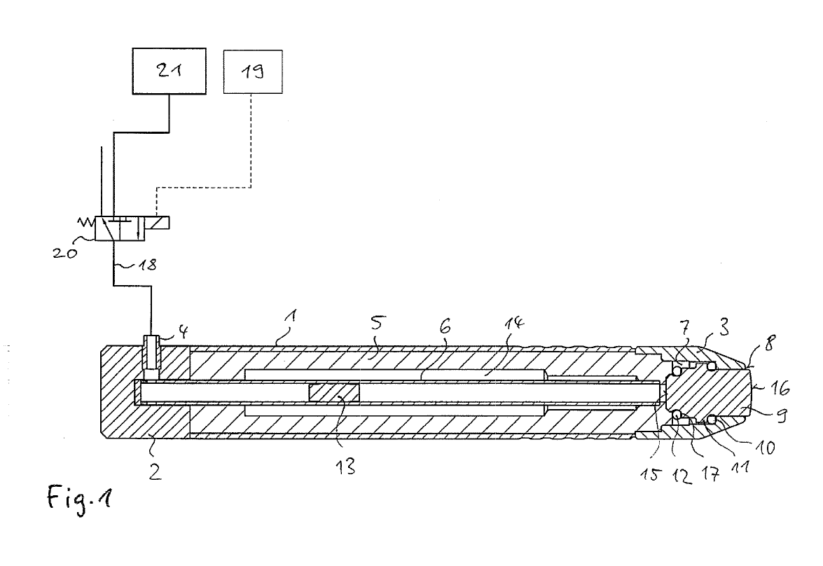

In FIG. 1, an apparatus according to the invention for coupling focused mechanical shockwaves, for example to a human body, is shown as a section along the longitudinal axis. A tube portion constitutes a casing 1 being closed at the respective ends by a air supply cap 2 distal from the body during application and an applicator cap 3 proximal to the body during application.

Air supply cap 2 comprises a pressurized air terminal 4 for a pneumatic supply. In a manner known as such, a valve 20 controlled by a control unit 19 via a pneumatic supply line 18 is connected to pressurized air terminal 4, in particular a magnetic valve, that couples in pressurized air pulses via the pressurized air terminal in a steady iteration rate between approximately 1 Hz and 50 Hz.

The apparatus is implemented as a device to be held manually by an operating person, which device is connected to a base station including control unit 19 and a compressor 21 by pneumatic line 18 mentioned and which can be positioned on the patient manually. It serves for the treatment of soft tissue, in particular muscles.

The details of the pneumatic supply are not relevant for this invention and are familiar to an expert as part of the prior art. Preferably, the frequency is adjustable. The iterative operation can be more complex than a simple steady repetition of pulses with a certain frequency, in particular also with a multiplicity of succeeding collisions in relatively short time distances, that is with a relatively high frequency, wherein groups of such collisions in these short time distances are mutually separated by somewhat longer time distances. Details of this aspect are not relevant for this invention but can be combined therewith.

A guiding tube 6 is supported within casing 1 by an insert 5 whose end distal from the body during application ends in air supply cap 2 and communicates with pressurized air terminal 4 there. The end of the guiding tube 6 proximal to the body during the application ends in a part of insert 5 projecting into applicator cap 3, namely short before the end of insert 5 there and an inner space 7 in applicator cap 3.

An impact body 9 is received in inner space 7 communicating with an applicator opening 8 being distal from the body during application, by two radial shoulders. Impact body 9 is supported on one of the radial shoulders by an O-ring 10 of an elastomer and has a flange 11 hereto. An end 15 of impact body 9 facing away from the body is supported on insert 5 by a further o-ring 12, namely on a front face encircling the already mentioned end of insert 5. Therein, O-ring 12 is positioned between this front face and a flange 17 or a shoulder of impact body 9. Applicator opening 8 serves for guiding impact body 9 in a manner displaceable in the longitudinal direction and fixes impact body 9 transverse to the longitudinal direction. The axial displaceability is limited by the resilience of elastomer rings 10 and 12 and is substantially higher than 0.6 mm relative to the rest of the apparatus in case of an operation of the apparatus in air.

The features of impact body 9 being the applicator to be positioned onto the skin will be discussed in further detail below.

A projectile 13 is inserted into the adjacent region of guiding tube 6 and is in contact with impact body 9 in FIG. 1. It fits radially with a small clearance (as regards the guiding tube and the substantially cylindrical geometry of projectile 13). Projectile 13 can be moved in guiding tube 6 by pressure differences of the air column in guiding tube 6 before and behind it (i. e. right and left of projectile 13 in FIG. 1), and can in particular be accelerated onto impact body 9. Hereto, it is accelerated from a starting position (not shown) in the left side in FIG. 1 by a pressurized air pulse via pressurized air terminal 4 and collides with impact body 9 by its front surface (not numerated in FIG. 1 for clarity of the drawing) facing impact body 9.

The back movement of projectile 13 is performed by a back flow of air from a pressure chamber 14 around guiding tube 6 within insert 5. During the acceleration of projectile 13 towards impact body 9, the air is pushed therein and compressed thereby. As soon as magnetic valve 20 in pneumatic line 18 of pressurized air terminal 4 switches off the pressure, projectile 13 is moved back into the starting position. Naturally, this can also be performed by an additional or alternative pressure application of pressure chamber 14 or another air volume on the body side of projectile 13. The end of guiding tube 6 distal from the body during application ends in a magnetic holder 17 for projectile 13.

Impact body 9 has a rotationally asymmetrical cylinder shape and is defined in the axial direction by the entry surface of end 15 and the somewhat convex exit surface 16. The outer circumferential surface has already described flange-like structures 11 and 17 providing support shoulders for O-rings 10 and 12. Further, an exit side part of the cylinder shape has a constant radius and is thus axially displaceable within opening 8.

According to the invention, impact body 9 consists of sintered ceramics, namely densified silicon nitride material (Si₃N₄). This is a polycrystalline material having a tetragonal crystal structure which has proven to be astonishingly impact resistant and hard. Quantitatively, the impact resistance is in the range of 6,500-7000 kJ/m² together with a pressure resistance of about 3,000 MPa.

This material is relatively light, namely, it has a density of 3.2 g/cm³. Since the sound velocity for longitudinal shockwaves is, although higher than in stainless steel, not too high, an acoustical impedance results which is around 20%-25% reduced and thus nearer to the acoustical impedance of the body tissue. Coupling-in of the shockwaves to the body tissue is thus even somewhat better than with conventional collision bodies.

Further, the material has a heat conductivity in the range of 20 W/mK and thus gives a less cold sensory impression than steel. It is tested as regards bio-compatibility.

In this regard, exemplary reference can be made to biological experiments as reported in “Biokompatibilität von Siliziumnitrid-Keramik in der Zellkultur. Eine vergleichende fluoreszenzmikroskopische und rasterelektronenmikroskopische Untersuchung”, Laryngo-Rhino-Otol 2004, 83: 845-851, also in Thieme-connect of the Georg Thieme Verlag and of Thieme Medical Publishers, Inc.

Finally, impact body 9 can be colored unproblematically (such as by addition of colored metal ions like Co) so that collision bodies of different form and/or different mass can be mounted for varying various treatment parameters, in particular the stroke, the size of exit surface 16, or the shape thereof, can be characterized by different colors, and can be mounted without the risk of mistakes. Hereto, the applicator cap can simply be screwed off.

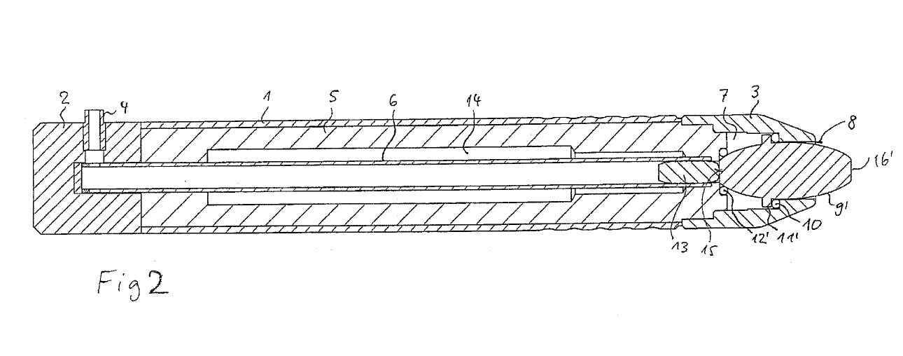

In FIG. 2, an analogous section through a second embodiment for coupling-in of focused mechanical shockwaves is shown. Predominately, the same reference numerals as in FIG. 1 have been used and will not be explained once more. Different therefrom is only the shape of an impact body 9′ as an ellipsoid of revolution.

Impact body 9′ is supported on one of the radial shoulders by an O-ring 10 of an elastomer and has a flange 11′ to this purpose. An end of impact body 9′ tapered towards the side distal from the body is supported on insert 5 by a further O-ring 12′, namely in a recess around an opening in the already mentioned end of insert 5.

The (originally) most convex left and right ends of the ellipsoid impact body 9′, respectively symmetrical to the longitudinal axis, are cut away in a straight manner, respectively. Therein, the left surface 15′ thus produced is a straight plane perpendicular to the longitudinal axis and runs through the focus of the ellipsoid there or of the ellipse shown in the section, respectively. The same applies to the right surface 16′ in that the “cut plane” runs through the second focus or more on the inside and has rounded edges, here.

Surface 16′ could also be positioned inside of the second focus so that the second focus would be positioned below the skin surface in the body tissue what is preferred in many cases.

The projectile front surface can also be somewhat convex in order to achieve a more point-like wave generation. Further, the stroke can be substantially smaller than 0.5 mm because here the component of the focused shockwave propagating through impact body 9′ is of interest.

1. An apparatus for treatment of biological body substances by being positioned onto a patient, said apparatus comprising
a moveable projectile (13) and
an impact body (9, 9′),
and being adapted to couple a mechanical shockwave to said biological body substance by accelerating said projectile (13) and colliding said projectile (13) onto said impact body (9, 9′),
wherein said impact body (9, 9′) is made of sintered ceramics.

2. The apparatus according to claim 1 wherein said ceramics consist of oxides, carbides, and/or nitrides by at least 80 weight-%.

3. The apparatus according to claim 1 wherein said impact body (9) has a cylindrical shape with an entry surface (15) on said projectile's side perpendicularly intersecting the cylinder axis and an exit surface (16) on said body substance's side perpendicularly intersecting said cylinder axis as well.

4. The apparatus according to claim 1 wherein said impact body (9′) has a circumferential surface portion being a part of an ellipsoid of revolution symmetrical to a longitudinal axis parallel to the direction of movement of said projectile (13),
wherein said impact body (9′) is shortened on said body substance's side (16′) at least up to the body substance's side focus of said ellipsoid of revolution and
is shortened on said projectile's side up to said projectile's side focus.

5. The apparatus according to claim 1 wherein said impact body (9, 9′) is supported in said apparatus by one or two elastomer rings (10, 12, 12′) and is guided in a manner axially displaceable against the elastic resistance of said elastomer ring(s) therein.

6. The apparatus according to claim 5 wherein said impact body (9, 9′) comprises at least one flange (11, 11′) for support against said elastomer ring(s) (10, 12, 12′).

7. The apparatus according to claim 1 having a pneumatic drive (4, 6, 10-20) for accelerating said projectile.

8. The apparatus according to claim 1 wherein said ceramics have a density of at most 6 g/cm³.

9. The apparatus according to claim 1 wherein said ceramics have an impact resistance of at least 3,000 kJ/m².

10. The apparatus according to claim 1 wherein said ceramics have a pressure resistance of at least 2,000 MPa.

11. The apparatus according to claim 1 wherein said ceramics are colored.

12. The apparatus according to claim 1 comprising a plurality of different collision bodies (9, 9′) being mountable by mutual exchange and distinguishable by different colors.

13. A method of use of the apparatus according to claim 1 for treating a soft body tissue.

14. The method according to claim 13, wherein said soft body tissue comprises body muscles and soft tissue regions near to the bones.

15. A method of use of the apparatus according to claim 11 wherein different collision bodies (9, 9′) are used and distinguishable by different colors.

16. A method for producing the apparatus according to claim 1 wherein said ceramic impact body (9, 9′) is pressed and tempered.
